(19) United States
(12) Patent Application Publication
(10) Pub. No.: US 2026/0151041 A1
(43) Pub. Date: Jun. 4, 2026

(54) Photoplethysmography (PPG) Sensor

(71) Applicants: TSLC Corporation, Chu-Nan (TW); Shin-Etsu Chemical Co., Ltd., Tokyo (JP); SCIVAX Corporation, Kawasaki City (JP)

(72) Inventors: Tomonori Nakamura, Mukai-cyo Tsurumi-ku (JP); CHEN-FU CHU, Hsinchu City (TW); TRUNG TRI DOAN, Hsinchu County (TW); SATORU TANAKA, Tokyo (JP)

(73) Assignees: TSLC Corporation, Chu-Nan (TW); Shin-Etsu Chemical Co., Ltd., Tokyo (JP); SCIVAX Corporation, Kawasaki City (JP)

(21) Appl. No.: 19/358,560

(22) Filed: Oct. 15, 2025

Related U.S. Application Data
(60) Provisional application No. 63/708,552, filed on Oct. 17, 2024.

(51) Int. Cl. A61B 5/024; A61B 34/20

(52) U.S. Cl. CPC A61B 5/02416; A61B 2034/2061; A61B 2562/185

(57) ABSTRACT

A photoplethysmography (PPG) sensor (30) includes an integrated package (32) having a light source (LS) device (34) configured to emit light (92) into biological tissue (38) of a user and a photodetector (PD) device (36) configured to receive reacted light transmitted through the biological tissue (38). The photoplethysmography (PPG) sensor (30) also includes metal interconnects (42) electrically connected to connection pads (40) on the light source (LS) device (34) and on the photodetector (PD) device (36), and an opaque covering structure (44) configured to enclose the light source (LS) device (34) and the photodetector (PD) device (36). The photoplethysmography (PPG) sensor (30) also includes a light guide structure (46) inside the opaque covering structure (44) having at least one non-opaque layer (50B) configured to guide the light emitted by the light source (LS) device (34).

### CROSS REFERENCE TO RELATED APPLICATION

This application claims priority from U.S. Provisional No. 63/708,552, filed Oct. 17, 2024, which is incorporated herein by reference.

### FIELD

This disclosure relates to photoplethysmography (PPG) sensors and to the fabrication of photoplethysmography (PPG) sensors and sensors using semiconductor fabrication techniques.

### BACKGROUND

Photoplethysmography (PPG) sensors are non-invasive optical devices that measure the change in blood volume in the skin's microvascular bed by detecting fluctuations in light absorption or reflection caused by each heartbeat. A conventional photoplethysmography (PPG) sensor includes a light source (LS) device and a photodetector (PD) device mounted on a printed circuit board (PCB). Photoplethysmography (PPG) sensors can monitor various physiological parameters, including heart rate, blood oxygen saturation, respiration, and blood pressure.

FIGS. 1A and 1B illustrate two different types of prior art photoplethysmography (PPG) sensors. In FIG. 1A, a prior art photoplethysmography (PPG) sensor 10 includes a light source (LS) device 16 configured to emit light, and a photodetector (PD) device 18 configured to detect light following transmission through biological tissue 12 (e.g., skin, blood vessels) on a body part (e.g., wrist, finger). The light source (LS) device 16 and the photodetector (PD) device 18 are both mounted to a printed circuit board (PCB) 14 with the photodetector (PD) device 18 configured to receive light that has reacted with the biological tissue 12.

In FIG. 1B, a prior art photoplethysmography (PPG) sensor 10M includes a sensor module 22M configured to contain a light source (LS) device 16M and a photo detector (PD) device 18M on a printed circuit board (PCB) 14M. The sensor module 22M includes a glass cover 20 configured to contact the biological tissue 12. The photodetector (PD) device 18M (FIG. 1B) also receives light reflected from the glass cover 20 that does not enter the biological tissue 12. This reflected light must be accounted for and can vary with the position of the sensor module 22M on the body part.

In the photoplethysmography (PPG) sensors 10 and 10M, the light undergoes various reactions with the biological tissue 12. The photodetector (PD) devices 18 and 18M are configured to detect the reacted light transmitted through the biological tissue 12 for monitoring selected physiological parameters. Some problems associated with prior art photoplethysmography (PPG) sensors include: the size of the (PPG) sensor limits a wearable system size, lower performance due to noises/crosstalk caused by stray light, and high costs due to packaging and mounting of a discrete light source (LS) devices and photodetector (PD) devices on a circuit board.

The present disclosure discloses photoplethysmography (PPG) sensors formed as integrated packages. These photoplethysmography (PPG) sensors overcome some of the problems associated with prior art (PPG) sensors including lower cost, smaller dimensions and reduced noise/crosstalk. The present disclosure is also directed to methods for fabricating photoplethysmography (PPG) sensors and sensors that integrate light source (LS) devices and photodetector (PD) devices into integrated packages using semiconductor fabrication techniques.

### SUMMARY

A photoplethysmography (PPG) sensor includes an integrated package comprising a light source (LS) device configured to emit light into biological tissue of a user and a photodetector (PD) device configured to receive reacted light transmitted through the biological tissue. The photoplethysmography (PPG) sensor also includes a plurality of metal interconnects physically and electrically connected to connection pads on the light source (LS) device and the photodetector (PD) device, and an opaque covering structure having an open end configured to enclose the light source (LS) device and the photodetector (PD) device on at least four sides.

The photoplethysmography (PPG) sensor also includes a light guide structure inside the opaque covering structure comprising at least one non-opaque layer configured to guide the light emitted by the light source (LS) device. The light guide structure includes a light escape area on the open end of the covering structure in light communication with the light source (LS) device, and a detection area on the open end of the opaque covering structure in light communication with the photodetector (PD) device. The non-opaque layers of the light guide structure can have different refractive indexes and can have the configuration of a distributed Bragg reflector (DBR) configured for light guiding. In addition, the light guide structure and the metal interconnects are configured to support the light source (LS) device, and the photodetector (PD) device without the requirement of a printed circuit board (PCB). In addition, the metal interconnects can include internal metal post interconnects embedded in the opaque covering structure and in the light guide structure.

In an alternate embodiment, a stacked photoplethysmography (PPG) sensor includes a light source (LS) device and a photodetector (PD) device in a stacked configuration configured to minimize noise/crosstalk. In the stacked photoplethysmography (PPG) sensor, the light source (LS) device can be mounted to a solid closed end of an opaque covering structure, and the photodetector (PD) device can be embedded in a light guide structure proximate to the open end of the opaque covering structure. The stacked photoplethysmography (PPG) sensor also includes a redistribution layer (RDL) on the light guide structure proximate to the open end of the opaque covering structure configured to provide circuit connections to internal interconnects in the opaque covering structure and to connection pads on the photodetector (PD) device.

The stacked photoplethysmography (PPG) sensor can also include a first dam on the light guide structure, which encloses the detection area and prevents stray light from the light source (LS) device, as well as other light sources, along with refracted and reflected stray light, from entering a detection area of the photodetector (PD) device. The first dam also prevents crosstalk between the light source (LS) device and the photodetector (PD) device, as it prevents light emitted by the light source (LS) device and transmitted through the light guide structure from entering the detection area on the light guide structure. The stacked photoplethysmography (PPG) sensor can also include a second dam on the detection area of the photodetector (PD) device, which is also configured to prevent stray light and crosstalk light from the light source (LS) device being detected by the photodetector (PD) device.

A photoplethysmography (PPG) array includes a criss-cross arrangement of multiple photoplethysmography (PPG) sensors electrically connected by interconnected addressed electrodes. Each discrete photoplethysmography (PPG) sensor is configured to form a closed region without interference such that clear 2D images can be obtained. The (PPG) array is particularly suited for vein authentication and brain wave measurement.

### BRIEF DESCRIPTION OF THE DRAWINGS

FIG. 1A is a schematic cross-sectional view of a prior art photoplethysmography (PPG) sensor;

FIG. 1B is a schematic cross-sectional view of a prior art photoplethysmography (PPG) sensor having a sensor module;

FIG. 2A is a schematic cross-sectional view of a photoplethysmography (PPG) sensor comprising an integrated chip scale package (CSP);

FIG. 2B is a schematic plan view of the photoplethysmography (PPG) sensor of FIG. 2A;

FIG. 3A is a schematic cross-sectional view of a stacked photoplethysmography (PPG) sensor having the photodetector (PD) device and the light source (LS) device in a stacked configuration configured to minimize noise/crosstalk;

FIG. 3B is a schematic top view of the stacked photoplethysmography (PPG) sensor shown in FIG. 3A;

FIG. 3C is a schematic bottom view of the stacked photoplethysmography (PPG) sensor shown in FIG. 3A;

FIG. 4A is a schematic cross-sectional view of a stacked photoplethysmography (PPG) sensor substantially identical to the stacked photoplethysmography (PPG) sensor shown in FIG. 3A but with a dam enclosing a detection area;

FIG. 4B is a schematic bottom view of the stacked photoplethysmography (PPG) sensor shown in FIG. 4A;

FIG. 5A is a schematic plan view of a (PPG) array constructed using multiple photoplethysmography (PPG) sensors as described in this disclosure;

FIG. 5B is a schematic cross-sectional view of the (PPG) array taken along section line 5B-5B of FIG. 5A;

FIG. 5C is an enlarged schematic cross sectional view of a single photoplethysmography (PPG) sensor of the (PPG) array taken along section line 5C of FIG. 5A;

FIG. 6A is a schematic cross-sectional view illustrating a photoplethysmography (PPG) sensor having a light source (LS) device and a photodetector (PD) device with a planar configuration and interconnected at the wafer level;

FIG. 6B is a schematic plan view of the photoplethysmography (PPG) sensor shown in FIG. 6A;

FIG. 6C is a schematic bottom view of the photoplethysmography (PPG) sensor shown in FIG. 6A;

FIG. 7 is a schematic cross-sectional view illustrating a photoplethysmography (PPG) sensor having four opaque sides;

FIG. 8 is a schematic cross-sectional view illustrating a photoplethysmography (PPG) sensor having conductive vias;

FIG. 9A is a schematic cross-sectional view illustrating a photoplethysmography (PPG) sensor having a second (PD) dam;

FIG. 9B is a schematic cross-sectional view illustrating a photoplethysmography (PPG) sensor having a second (PD) and conductive vias;

FIG. 10 is a schematic cross-sectional view illustrating a photoplethysmography (PPG) sensor having a second (PD) dam;

FIG. 11 is a schematic cross-sectional view illustrating a photoplethysmography (PPG) sensor having a (PD) covering material;

FIG. 12A is a schematic cross-sectional view illustrating a photoplethysmography (PPG) sensor having covering material between a light source (LS) device and a photodetector (PD) device;

FIG. 12B is a schematic plan view of the photoplethysmography (PPG) sensor shown in FIG. 12A;

FIG. 12C is a schematic bottom view of the photoplethysmography (PPG) sensor shown in FIG. 12A;

FIG. 13A is a schematic cross-sectional view illustrating a multiple zone flip chip (LED);

FIG. 13B is a schematic cross-sectional view illustrating a photoplethysmography (PPG) sensor that includes a multiple zone light source (LS) device fabricated using the multiple zone flip chip (LED);

FIG. 14 is a schematic cross-sectional view illustrating a photoplethysmography (PPG) sensor that includes a multiple light source (LS) device paired with multiple photodetector (PD) devices;

FIG. 15A is a schematic cross-sectional view illustrating a multiple color (LED) module;

FIG. 15B is a schematic cross-sectional view illustrating a photoplethysmography (PPG) sensor that includes a multiple zone light source (LS) device fabricated using the multiple color (LED) module;

FIG. 16 is a schematic cross-sectional view illustrating a photoplethysmography (PPG) sensor that includes a multiple color (LED) module paired with multiple photodetector (PD) devices;

FIGS. 17A-17L are schematic cross-sectional views illustrating a sensor configured to detect pressure having a side-by-side configuration and a membrane light guide structure with a variable thickness that can be changed as a function of pressure for varying a light intensity of reflected light;

FIGS. 18A-18B are schematic cross-sectional views illustrating a sensor configured to detect pressure having a side-by-side configuration and a reflector on the membrane light guide structure; and

FIGS. 19A-19B are schematic cross-sectional views illustrating a sensor configured to detect pressure having a stacked configuration and a reflector on the membrane light guide structure.

### DETAILED DESCRIPTION

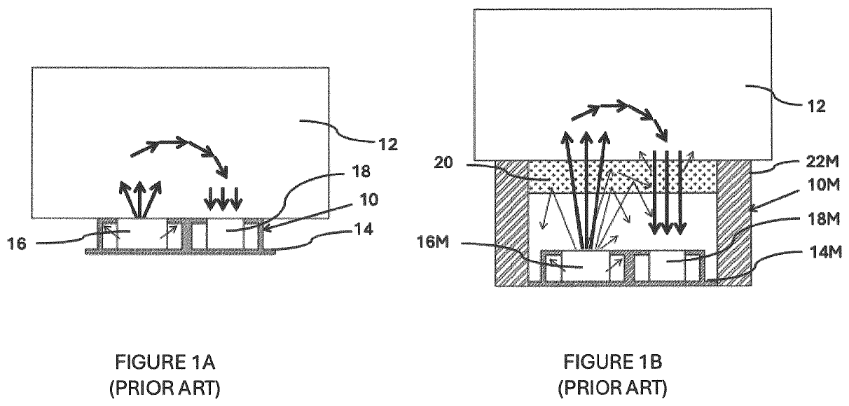

Referring to FIGS. 1A and 1B, a photoplethysmography (PPG) sensor 30 is shown. The photoplethysmography (PPG) sensor 30 includes an integrated chip scale package (CSP) 32 comprising a light source (LS) device 34 configured to emit light into biological tissue 38 of a user and a photodetector (PD) device 36 configured to receive reacted light transmitted through the biological tissue 38. By way of example, the biological tissue 38 can comprise skin, blood vessels, organs, muscles as well as other biological systems on a user. In addition, the photoplethysmography (PPG) sensor 30 can be used with wearable devices such as smartwatches, fitness trackers, and clinical monitoring systems to provide continuous health data on the user.

The light source (LS) device 34 can be configured to emit light having a desired wavelength selected as a function of the biological tissue 38 and the physiological parameters being sensed. Exemplary wavelengths include green (around 523-532 nm), red (around 660 nm), and near-infrared (NIR, around 800-940 nm). Different wavelengths can be used to probe different layers of biological tissue 38, such as green light for superficial skin and longer NIR wavelengths for deeper arteries.

The light source (LS) device 34 can comprise a packaged or unpackaged (LED) die having a plurality of connection pads 40 that can include a VDD connection pad 40VDD and a VDS connection pad 40VDS. In the examples to follow, the light source (LS) device 34 comprises a light emitting diode (LED) die (e.g., a vertical light emitting die (VLED), a planar (PLED) or a multiple wavelength (LED)). In addition, the integrated package has the configuration of a package on package (POP) fan out chip scale package (FOCSP) having a (VCSEL) on driver (VoD) electrical configuration. However, other types of light emitting devices can also be used as the light source (LS) device 34. Other suitable light source (LS) devices 34 include VCSEL (Vertical-Cavity Surface-Emitting Laser) and EEL (Edge Emitting Laser) devices, or any type of light emitting device having cathode and anode connection pads.

The photoplethysmography (PPG) sensor 30 also includes a plurality of metal post interconnects 42 physically and electrically connected to the connection pads 40 on the light source (LS) device 34 and on the photodetector (PD) device 36. By way of example, the photodetector (PD) device 36 can include an anode connection pad 40A and a cathode connection pad 40C. Preferably the metal post interconnects 42 are configured such that the photodetector (PD) device 36 and the light source (LS) device 34 can be operated and turned on and off independently.

The photoplethysmography (PPG) sensor 30 also includes an opaque covering structure 44 having an open end 64 and a solid closed end 68. Suitable materials for forming the opaque covering structure 44 include silicone, epoxy, glue, gel, BCB, polyimide, SOG, PDMS, as well as other opaque organic materials that have been treated with additives if necessary to be opaque to light at all wavelength ranges. The opaque covering structure 44 can comprise a hollow box-like structure having polygonal sides (e.g., square, rectangular) configured to enclose the light source (LS) device 34 and the photodetector (PD) device 36 on at least four sides. In the illustrative embodiment, the box-like structure has six sides total, with the open end 64 being the sixth side, which is not covered by the opaque material that forms the opaque covering structure 44, such that five sides are covered. Alternately, other polygonal configurations can be used for the opaque covering structure 44.

The photoplethysmography (PPG) sensor 30 also includes a light guide structure 46 inside an interior portion of the opaque covering structure 44. The light guide structure 46 can comprise multiple layers of non-opaque materials having different refractive indexes configured as a distributed Bragg reflector (DBR). In addition, the light guide structure 46 can include a lens 48 and one or more non-opaque layers 50A, 50B, 50C configured to guide the light emitted by the light source (LS) device 34, and the reacted light transmitted to the photodetector (PD) device 36. Suitable materials for forming the non-opaque layers 50A, 50B, 50C include silicone, epoxy, glue, gel, BCB, polyimide, SOG, PDMS, as well as other non-opaque organic materials, which have been treated with additives if necessary, to be non-opaque to light at all wavelength ranges.

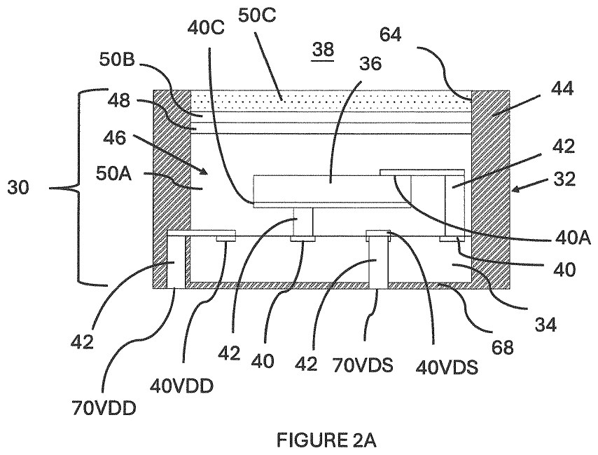

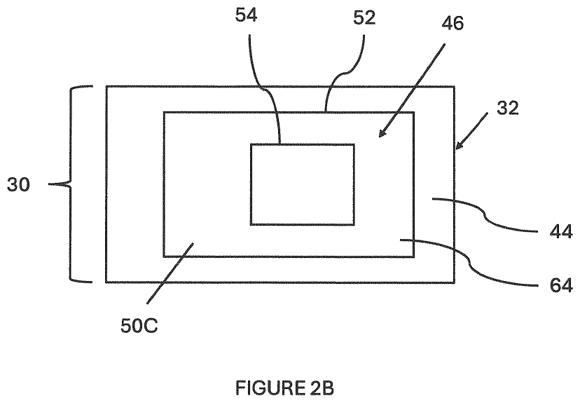

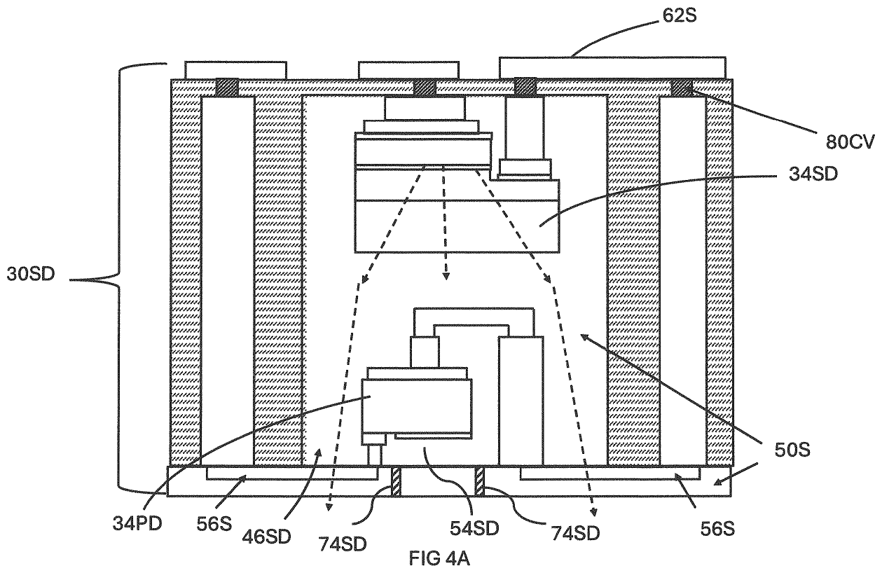

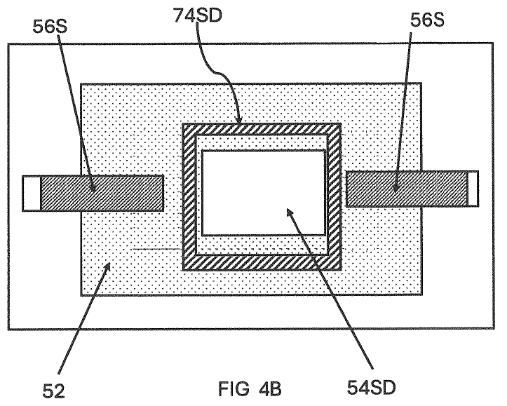

As shown in FIG. 2B, the light guide structure 46 includes a light escape area 52 on the open end 64 of the covering structure 44 in light communication with the light source (LS) device 34, and a detection area 54 on the open end 64 of the covering structure 44 in light communication with the photodetector (PD) device 36. In addition, the light guide structure 46 and the metal post interconnects 42 are configured to support the light source (LS) device 34, and the photodetector (PD) device 36 without the requirement of a printed circuit board (PCB). Further, the closed end 68 of the covering structure 44 provides a surface for mounting circuit external contacts such as a VDD contact 70VDD and VDS contact 70VDS. As will be further explained, the light guide structure 46 can also include a dam 74SD (FIG. 4B) configured to prevent stray light from entering the detection area 54.

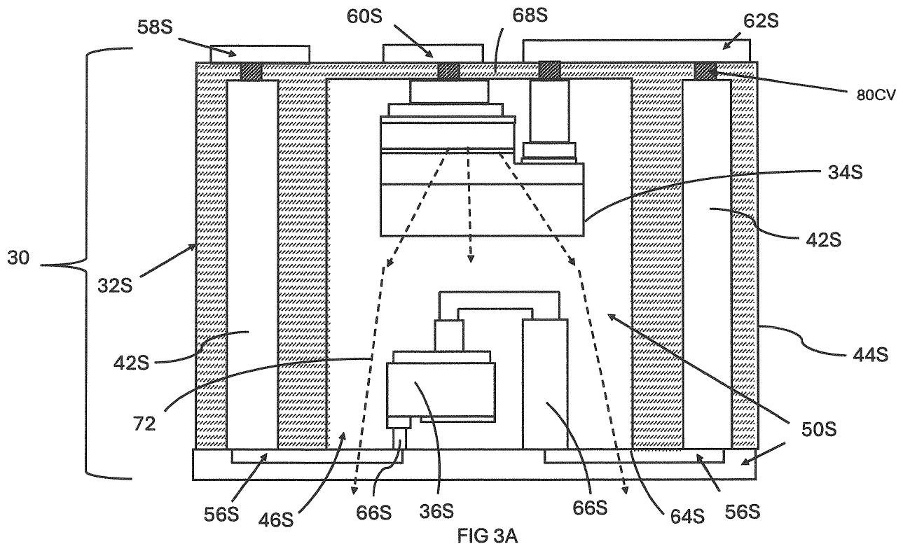

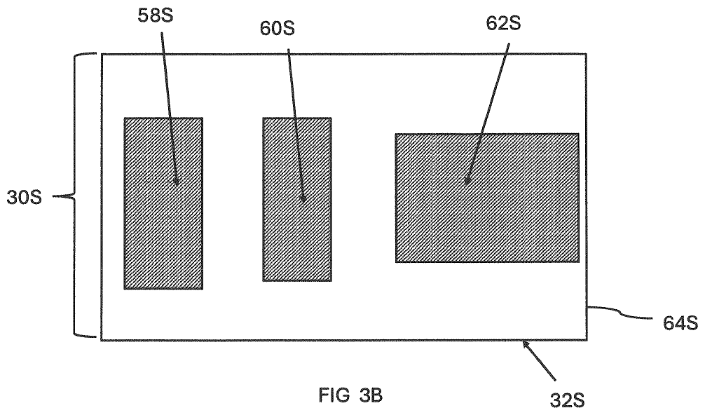

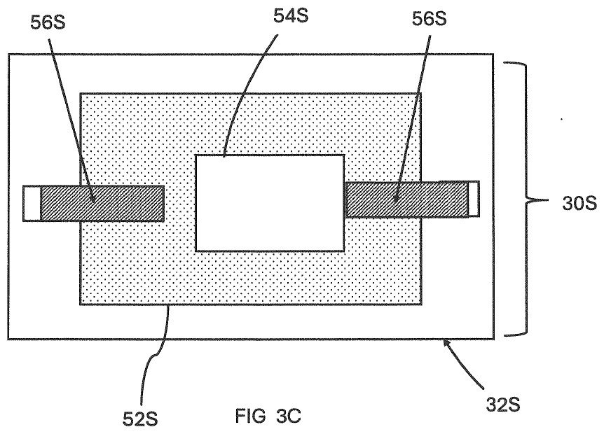

Referring to FIGS. 3A-3C, a stacked photoplethysmography (PPG) sensor 30S includes the light source (LS) device 34S and the photodetector (PD) device 36S in a stacked configuration configured to minimize noise/crosstalk. In addition to being stacked, the optical axes of the light source (LS) device 34S and the photodetector (PD) device 36S are preferably in substantially alignment with one another and more preferably coincident to one another. The light source (LS) device 34S comprises a packaged vertical light emitting diode (VLED) die configured to emit light 72 having a desired intensity and wavelength. This type of packaged vertical light emitting diode (VLED) die is known in the art and is commercially available from manufacturers such as TSLC Corporation of Chu-nan, Taiwan.

The photoplethysmography (PPG) sensor 30S also includes an opaque covering structure 44S having an open end 64S, substantially as previously described. As shown in FIG. 3C, the open end 64S of the covering structure 44S includes a light escape area 52S and a light detection area 54S on a light guide structure 46S, substantially as previously described. The opaque covering structure 44S also includes internal metal post interconnects 42S which rigidify the structure and provide electrical paths through the opaque covering structure 44S. The opaque covering structure 44S also includes a closed end 68S formed of solid material substantially as previously described. In the photoplethysmography (PPG) sensor 30S, the light source (LS) device 34S is mounted to the closed end 68S of the opaque covering structure 44S, and the photodetector (PD) device 36S is embedded in the light guide structure 46S proximate to the open end 64S of the opaque covering structure 44S.

Still referring to FIGS. 3A-3C, the photoplethysmography (PPG) sensor 30S also includes a redistribution layer (RDL) 56S proximate to the open end 64S of the opaque covering structure 44S formed on the surface of the light guide structure 46S. The redistribution layer (RDL) 56S includes circuit traces in electrical communication with the internal metal post interconnects 42S and with metal posts 66S embedded in the light guide structure 46S. In addition to providing a support function, the metal posts 66S are in electrical contact with corresponding connection pads on the photodetector (PD) device 36S substantially as previously described. The internal metal post interconnects 42S are also in physical and electrical contact with external device pads that can include an external photodetector (PD) device anode pad 58S, an external light source (LS) device anode pad 60S, and an external common cathode pad 62S formed on the closed end 68S of the opaque covering structure 44S. These external device pads 58S, 60S, 62S are also in electrical communication with corresponding connection pads on the light source (LS) device 34S, substantially as previously described. In addition, the redistribution layer (RDL) 56S, the metal post interconnects 42S, and the external device pads are 58S, 60S, 62S are configured such that the light source (LS) device 34S and the photodetector (PD) device 36S can be turned on and off separately. Also in this embodiment, and in all of the embodiments to follow, the common cathode pad 62S can have other configurations, such as separate pads and circuits for the light source (LS) device 34S and the photodetector (PD) device 36S.

Referring to FIGS. 4A-4B, a photoplethysmography (PPG) sensor 30SD is substantially identical to the photoplethysmography (PPG) sensor 30S (FIG. 3A) but also includes a dam 74SD enclosing a detection area 54SD. In the illustrative embodiment, the dam is embedded in the light guide structure 46SD. The dam 74SD can comprise a metal or polymer material formed using a suitable method such as PVD, CVD, sputtering, electro-plating, molding, photo lithography, laser patterning, dispensing, inkjet printing or screen printing. The dam 74SD is configured to prevent stray light and crosstalk light from entering the detection area 54SD of the light guide structure 46SD. Although the light guide structure 46SD is formed of optical quality planarized materials, some stray light, such as refracted and reflected light, may be present. However, this stray light is prevented from entering a detection area on the photodetector (PD) device 36SD by the dam 74SD. The dam 74SD also substantially reduces or eliminates crosstalk light from the light source (LS) device 34SD entering the photodetector (PD) device 36SD. As used herein the term “crosstalk” means unwanted light communication between the light source (LS) device 34SD and the photodetector (PD) device 36SD.

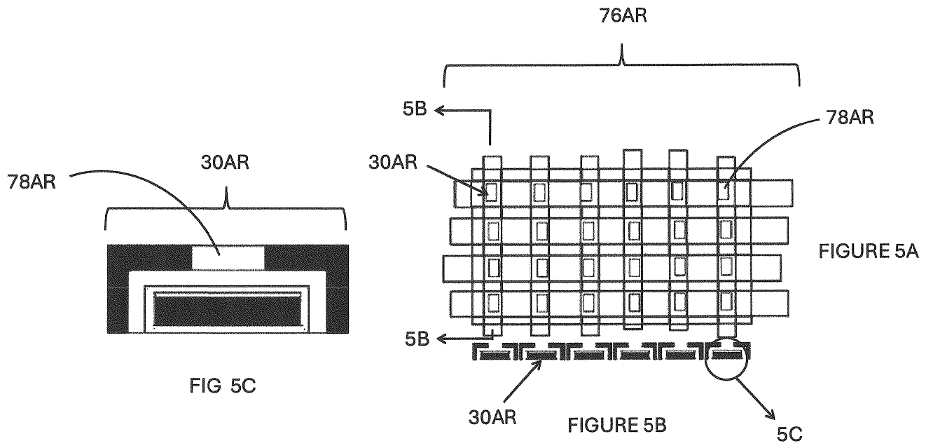

Referring to FIGS. 5A-5C, a photoplethysmography (PPG) array 76AR is illustrated. The photoplethysmography (PPG) array 76AR comprises a criss-cross arrangement of multiple photoplethysmography (PPG) sensors 30AR constructed as previously described for any of the embodiments. The photoplethysmography (PPG) sensors 30AR can be arrayed efficiently and driven by interconnected addressed electrodes 78AR. Each discrete photoplethysmography (PPG) sensor 30AR is configured to from a closed region without interference such that clear 2D images can be obtained. The photoplethysmography (PPG) sensors 30AR provide the smallest size and footprint for the photoplethysmography (PPG) array 76AR. The photoplethysmography (PPG) array 76AR is particularly suited for vein authentication and brain wave measurement.

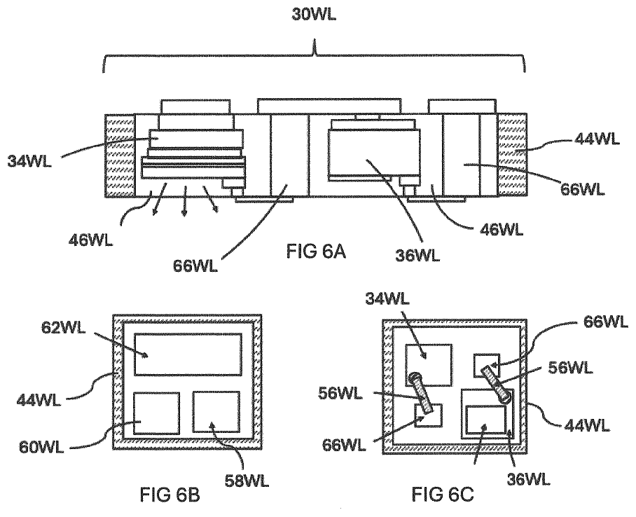

Referring to FIGS. 6A-6C, a photoplethysmography (PPG) sensor 30WL fabricated using wafer level (WL) fabrication techniques is illustrated. The photoplethysmography (PPG) sensor 30WL includes a light source (LS) device 34WL and a photodetector (PD) device 36WL and an opaque covering structure 44WL. In this embodiment the light source (LS) device 34WL and the photodetector (PD) device 36WL are embedded in a light guide structure 46WL and mounted side by side in a planar configuration. Rather than just one light source (LS) device 34WL and the photodetector (PD) device 36WL there can be any number of these devices in an array. The light source (LS) device 34WL can also comprise a laser light source. A redistribution layer (RDL) 56WL and metal posts 66WL electrically connect a (PD) device anode pad 58WL, a (LS) device anode pad 60WL and a common cathode pad 62WL. The metal posts 66WL, the redistribution layer (RDL) 56WL, the (PD) device anode pad 58WL and the (LS) device anode pad 60WL can be formed using wafer level fabrication processes selected from the group consisting of electrical plating with lithography patterning, PVD, CVD, damascene, and laser ablation. After applying suitable materials (e.g., polymers) for forming the light guide structure 46WL and the light guide structure 46WL, planarization of the light guide structure 46WL can be performed (if needed) using a process selected from the group consisting of chemical mechanical polishing (CMP), mechanical polishing/grinding and etch back planarization. As in the previous embodiments the light guide structure 46WL comprises a non-opaque transparent material and the opaque covering structure 44WL comprises an opaque material substantially as previously described. The light guide structure 46WL and the opaque covering structure 44WL can comprise a material selected from the group consisting of silicone, epoxy, glue, gel, BCB, polyimide, PDMS and other organic materials.

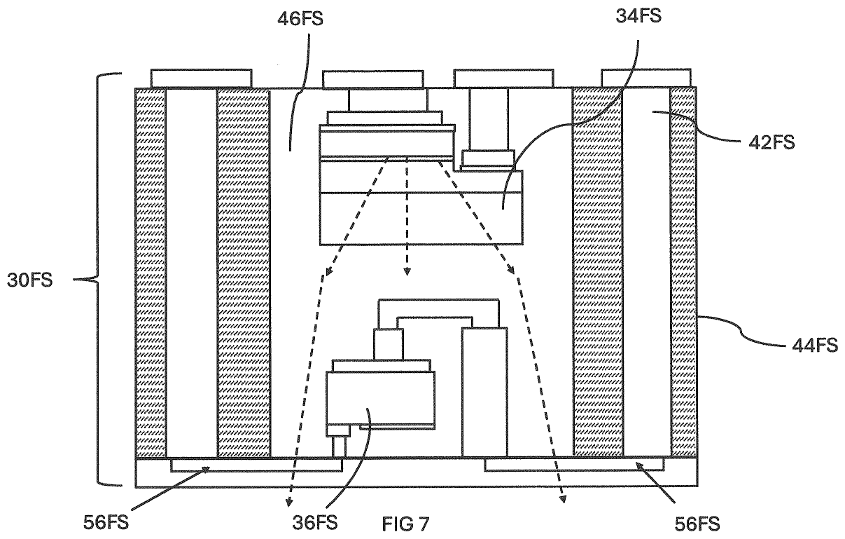

Referring to FIG. 7, a photoplethysmography (PPG) sensor 30FS having a four sided opaque covering structure 44FS is illustrated. Metal post interconnects 42FS and redistribution layer (RDL) 56FS can be fabricated using a process selected from the group consisting of electrical plating with lithography patterning, PVD, CVD, damascene and laser metal ablation. In this embodiment, the electrical connections are substantially as previously described. In addition, the light source (LS) device 34FS and the photodetector (PD) device 36FS are embedded in and supported by the light guide structure 46FS.

Referring to FIG. 8, a photoplethysmography (PPG) sensor 30CV having conductive vias 80CV is illustrated. This embodiment is similar to photoplethysmography (PPG) sensor 30S (FIG. 3A). The conductive vias 80CV can be formed using a process selected from the group consisting of electrical plating with lithography patterning, PVD, CVD, damascene and laser metal ablation.

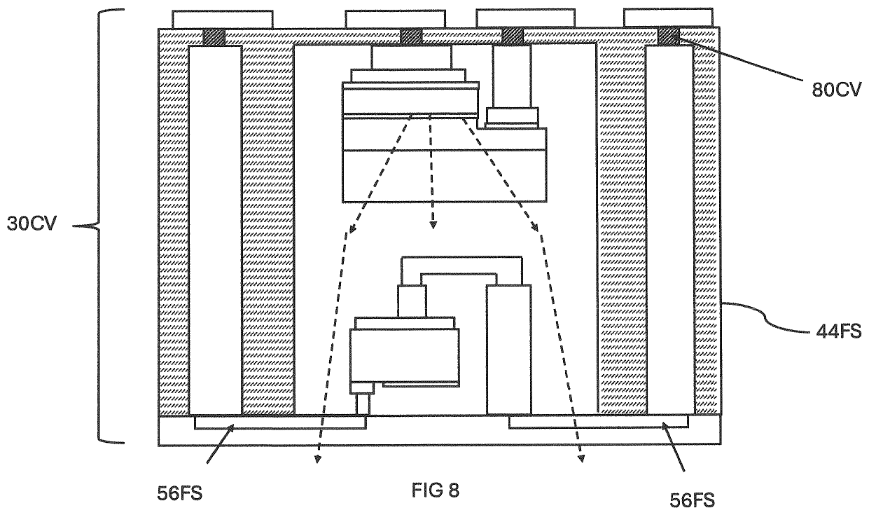

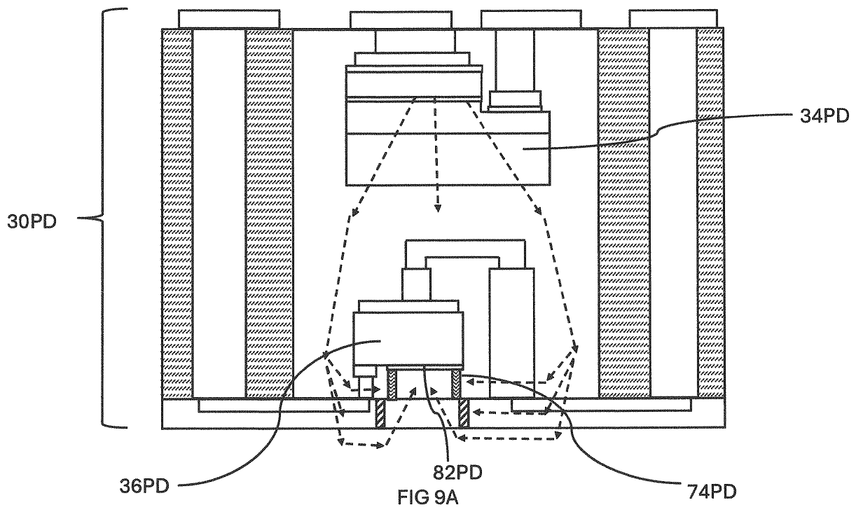

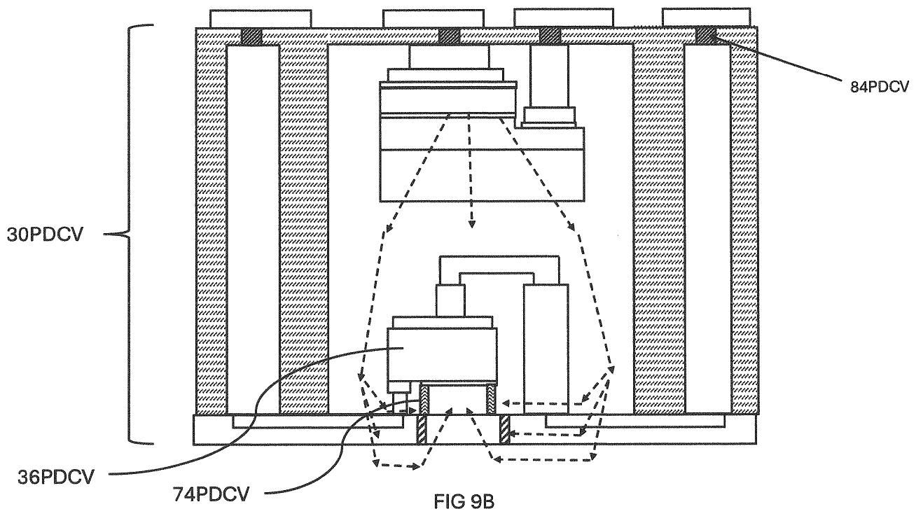

Referring to FIG. 9A, a photoplethysmography (PPG) sensor 30PD having a second (PD) dam 74PD, which surrounds a detection area 82PD on the photodetector (PD) device 36PD, is illustrated. This embodiment is substantially identical to photoplethysmography (PPG) sensor 30SD (FIG. 4A) but also includes the second (PD) dam 74PD. As indicated by the light arrows, the second (PD) dam 74PD only lets reacted light rays into the photodetector (PD) device 36PD and prevents stray light from entering. In addition, the second (PD) dam 74PD also prevents crosstalk between the photodetector (PD) device 36PD and the light source (LS) device 34PD. The second (PD) dam 74PD can be formed substantially as previously described for dam 74SD (FIG. 4A), but rather than being formed on the light guide structure 46SD (FIG. 4A), it is formed directly on the photodetector (PD) device 36PD (FIG. 9A).

Referring to FIG. 9B, a photoplethysmography (PPG) sensor 30PDCV having a second (PD) dam 74PDCV on the photodetector (PD) device 36PD and conductive vias 84PDCV is illustrated. This embodiment is substantially identical to photoplethysmography (PPG) sensor 30CV (FIG. 8). As indicated by the light arrows, the second (PD) dam 74PDCV only lets reacted light rays into the photodetector (PD) device 36PDCV and prevents stray light from entering. In addition, the second (PD) dam 74PDCV prevents crosstalk as previously described. The second (PD) dam 74PDCV can be formed substantially as previously described for dam 74SD (FIG. 4A). The conductive vias 84PDCV can be formed substantially as previously described for conductive vias 80CV (FIG. 8).

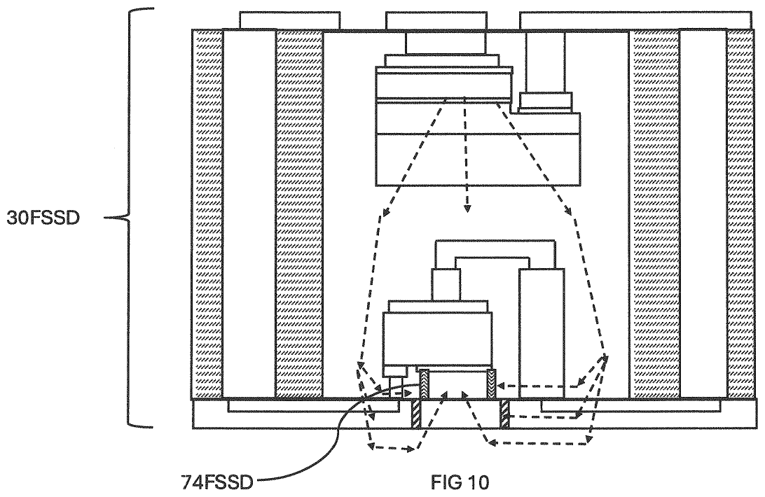

Referring to FIG. 10, a photoplethysmography (PPG) sensor 30FSSD is substantially similar to photoplethysmography (PPG) sensor 30FS (FIG. 7) but includes a second (PD) dam 74FSSD substantially as previously described.

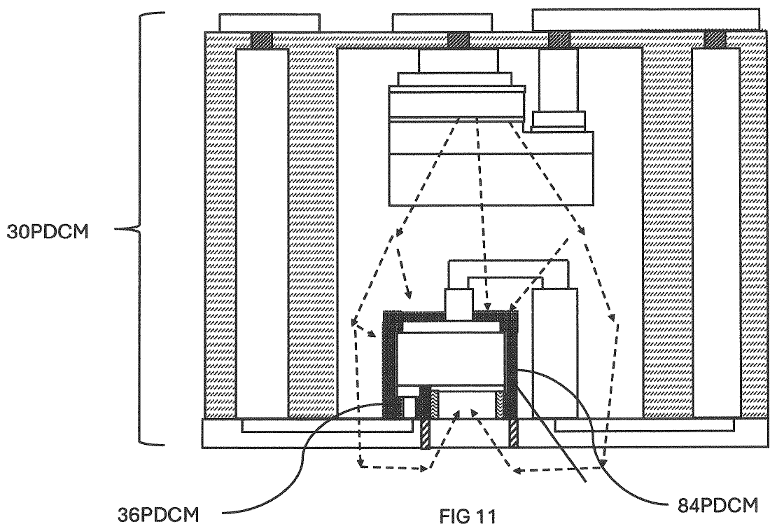

Referring to FIG. 11, a photoplethysmography (PPG) sensor 30PDCM is substantially similar to photoplethysmography (PPG) sensor 30CV (FIG. 8) but includes a covering material 84PDCM encapsulating a photodetector (PD) device 36PDCM. The covering material 84PDCM shields the photodetector (PD) device 36PDCM and prevents crosstalk substantially as previously described. The covering material 84PDCM can comprise an opaque material as previously described for opaque covering structure 44 (FIG. 2A).

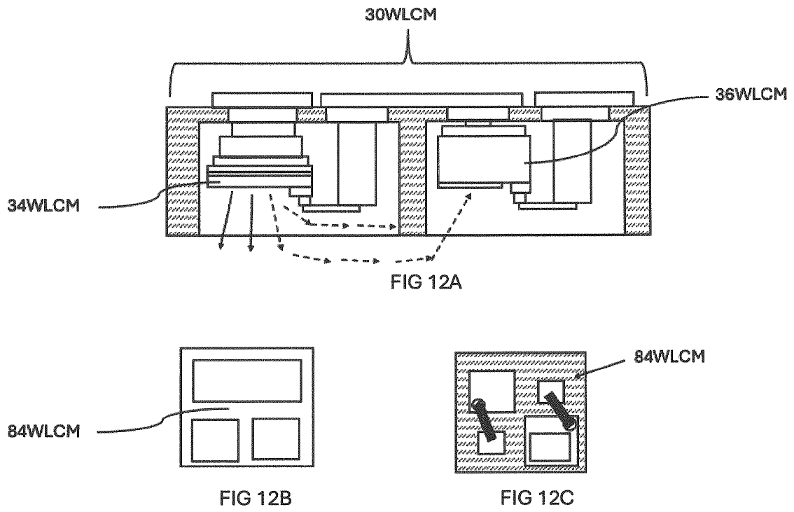

Referring to FIGS. 12A-12C, a photoplethysmography (PPG) sensor 30WLCM is substantially similar to photoplethysmography (PPG) sensor 30WL (FIG. 6A) but includes internal covering material 84WLCM between the light source (LS) device 34WLCM the photodetector (PD) device 36WLCM. The internal covering material 84WLCM prevents crosstalk between the light source (LS) device 34WLCM and the photodetector (PD) device 36WLCM. The covering material 84WLCM can comprise an opaque material as previously described for opaque covering structure 44 (FIG. 2A).

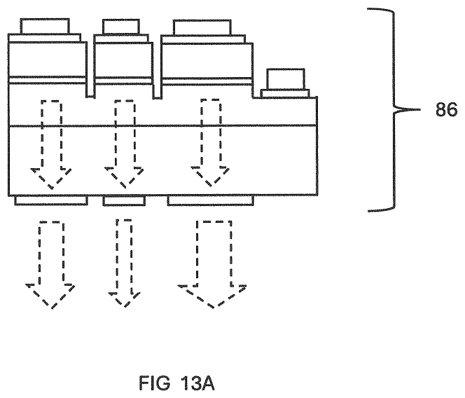

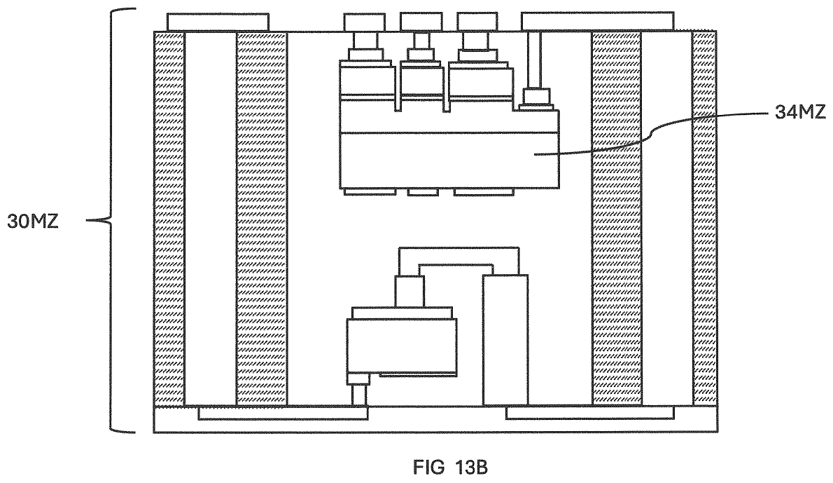

Referring to FIG. 13A, a multiple zone flip chip (LED) 86 is shown. The multiple zone flip chip (LED) 86 includes multiple light emitting zones as described in U.S. Pat. No. 10,964,851 B2, Single Light Emitting Diode (LED) Structure, which is incorporated herein by reference. Referring to FIG. 13B, the multiple zone flip chip (LED) 86 can be used to fabricate a photoplethysmography (PPG) sensor 30MZ having a multiple zone light source (LS) device 34MZ. Each zone can emit a different wavelength of light permitting multiple physiological parameters to be detected at the same time with a single photoplethysmography (PPG) sensor 30MZ.

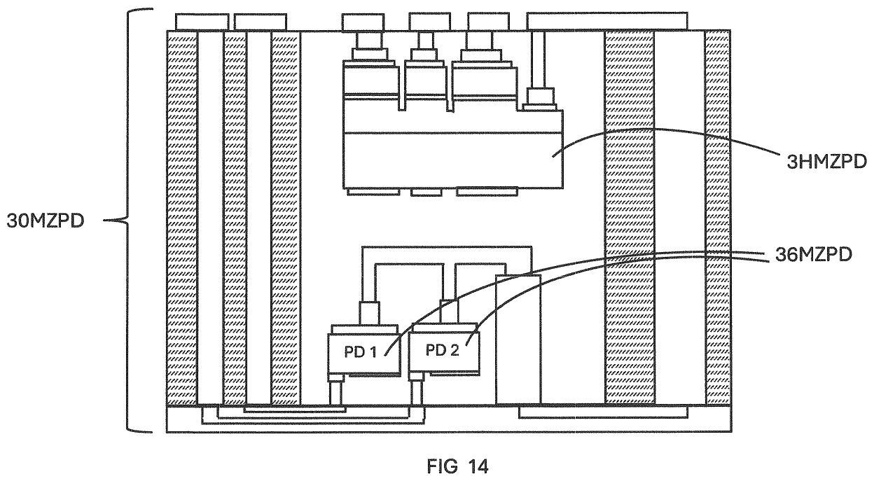

Referring to FIG. 14, a photoplethysmography (PPG) sensor 30MZPD includes a multiple zone light source (LS) device 34MZPD paired with multiple photodetector (PD) devices 36MZPD. Each zone can have a different wavelength for emitted light permitting multiple physiological parameters to be detected at the same time with a single photoplethysmography (PPG) sensor 30MZ. Each zone can emit a different wavelength of light and each photodetector (PD) devices 36MZPD can detect a particular wavelength permitting multiple physiological parameters to be detected at the same time with a single photoplethysmography (PPG) sensor 30MZPD.

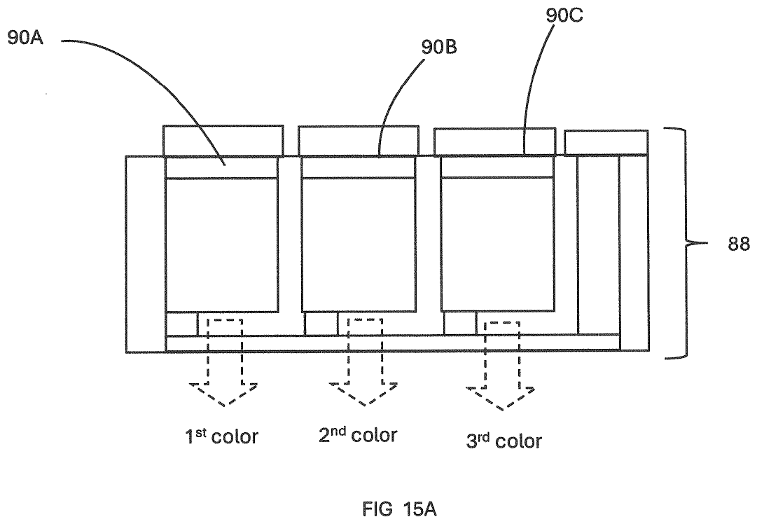

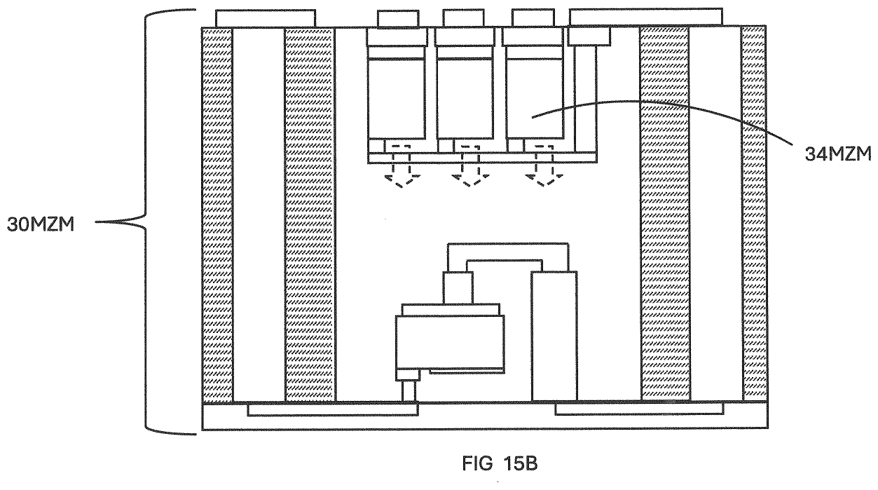

Referring to FIG. 15A, a multiple color (LED) module 88 is shown. The multiple color (LED) module 88 includes separate color LEDs 90A, 90B 90C, each of which is configured to emit light of a different wavelength. Referring to FIG. 15B, the multiple color (LED) module 88 can be used to fabricate a photoplethysmography (PPG) sensor 30MZM having a multiple color light source (LS) device 34MZM. Each color comprises a different wavelength of light permitting multiple physiological parameters to be detected at the same time with a single photoplethysmography (PPG) sensor 30MZM.

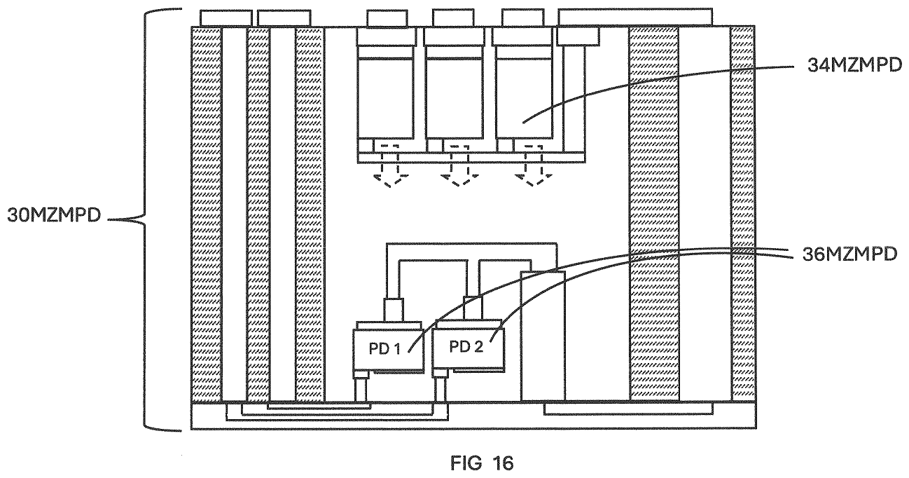

Referring to FIG. 16, a photoplethysmography (PPG) sensor 30MZMPD includes a multiple color light source (LS) device 34MZMPD paired with multiple photodetector (PD) devices 36MZMPD. Each color comprises a different wavelength for emitted light permitting multiple physiological parameters to be detected at the same time with a single photoplethysmography (PPG) sensor 30MZMPD.

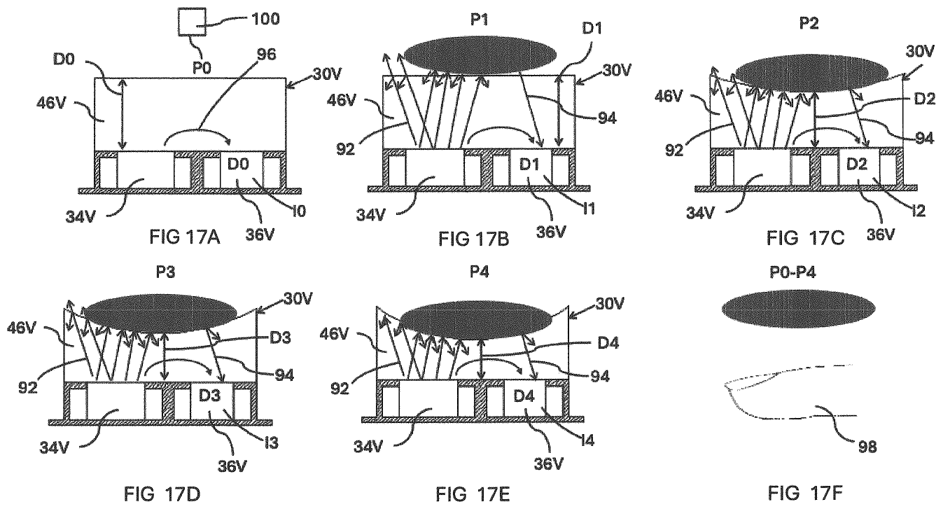

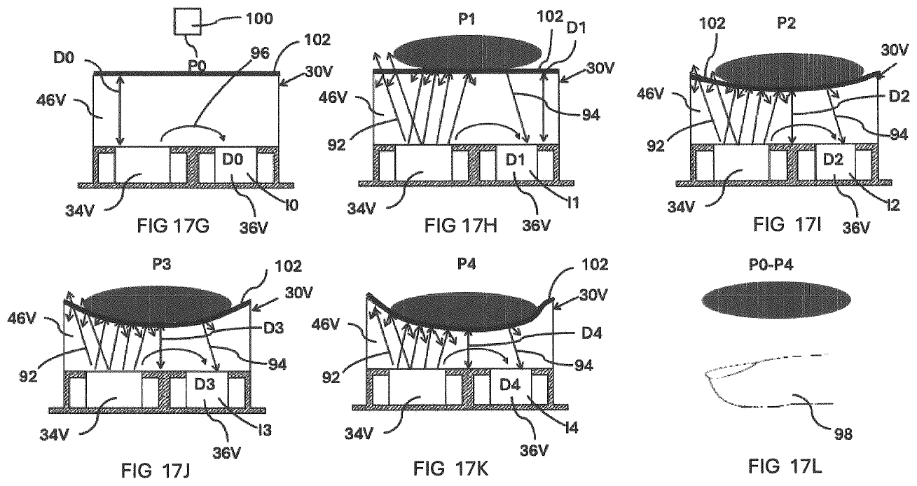

Referring to FIGS. 17A-17F, a sensor 30V is substantially similar to the photoplethysmography (PPG) sensor 30WL shown in FIG. 6A. The sensor 30V is configured to detect pressure. The sensor 30V can be also configured to detect vibration and fluid flow. The sensor 30V includes a light source (LS) device 34V for emitting light 92 and a photodetector (PD) device 36V for detecting reflected light 94 in a side-by-side configuration as previously described. The sensor 30V also includes a membrane light guide structure 46V having a variable thickness D0-D4 that can be changed as a function of pressure P0-P4 for varying the intensity of the reflected light 94 detected by the photodetector (PD) device 36V. In addition, crosstalk light 96 between the light source (LS) device 34V and the photodetector (PD) device 36V can also occur and is indicated by the horizontal vector. As shown in FIGS. 17G-17L, the sensor 30V can also include a reflector 102 formed on the exterior surface of the membrane light guide structure 46V. Further details of the reflector 102 will become more apparent as the description proceeds.

As shown in FIG. 17B, the membrane light guide structure 46V can comprise a semitransparent or transparent material configured for light transmission therethrough, with semitransparent being preferred. In addition, the membrane light guide structure 46V can comprise an elastic, compressible polymer layer such as an elastic compound material, such as silicone, formed on the previously described photoplethysmography (PPG) sensor 30WL (FIG. 6A). As shown in FIG. 17G, a reflector 102 can be formed on top of the membrane light guide structure 46V. The reflector 102 can comprise a material having Young's modulus (E) selected such that the reflector 102 remains elastic when a known load is applied. Young's modulus (E), also known as the modulus of elasticity, quantifies the relationship between tensile or compressive stress (force per unit area) and the resulting strain (deformation) in a material. It is fundamental property that describes how much a material will deform under a given load. It is important that under the maximum force, the reflector 102 still operates in the elastic regime and are not plastically deformed. The reflector 102 can be a single layer of multilayer of reflective materials depending on the application, such as titanium/aluminum, molybdenum/gold, tungsten/titanium stacking layers.

As shown in FIG. 17B, in the sensor 30V, some of the emitted light 92 from the light source (LS) device 34V can be reflected to the photodetector (PD) device 36V as reflected light 94. As shown in FIGS. 17B-17E, a pressure press P1-P4 on the membrane light guide structure 46V changes the thicknesses D0-D4 such that the reflected light 94 increases along with the light intensity thereof. As shown in FIG. 17F, the pressure PO-P4 can be generated by a finger 98 of a user of the sensor 30V. Different pressure forces P0-P4 changes reflected light intensities as the light reflection paths can be increased or decreased with the distances DO-D4 corresponding to the variable thicknesses of the membrane light guide structure 46V.

In FIG. 17A, the reflected light 94 has an initial intensity 10 and P0 indicates no pressure source being exerted on the membrane light guide structure 46V. FIG. 17B shows a pressure source PS01 having a pressure force P1 being exerted on the membrane light guide structure 46V, which has been deformed by the pressure force P1 to distance D1. FIG. 17C shows an increased pressure force P2 in contact with the membrane light guide structure 46V, which has been deformed by the pressure force P2 to distance D2. FIG. 17D shows an increased pressure force P3 which further deforms the membrane light guide structure 46V to distance D3. FIG. 17E shows an even greater pressure force P4, which further deforms the membrane light guide structure 46V to distance D4.

The emitted light 92 can be reflected and scattered through the membrane light guide structure 46V such that the photodetector (PD) device 36V detects more light intensity 14 in FIG. 17E than that of the I3 of FIG. 17D. Exemplary pressure forces as a function of distances can include P0 corresponding to D0, P1 corresponding to D1, P2′ corresponding to D2, P3 corresponding to D3, P4 corresponding to D4. Exemplary relationships for the intensity 10-14 of the reflected light 94 include: P0<P1<P2<P3<P4 and I0<I1<I2<I3<I4. During operation of the sensor 30V, a pressure sensor 100 can be used to detect the pressure force strength P0-P4 by reading the intensity value 10-14 of reflected light 94 at the photodetector (PD) device 36V. FIGS. 17G-17K show substantially the same operation of the sensor 30V but with the reflector 102.

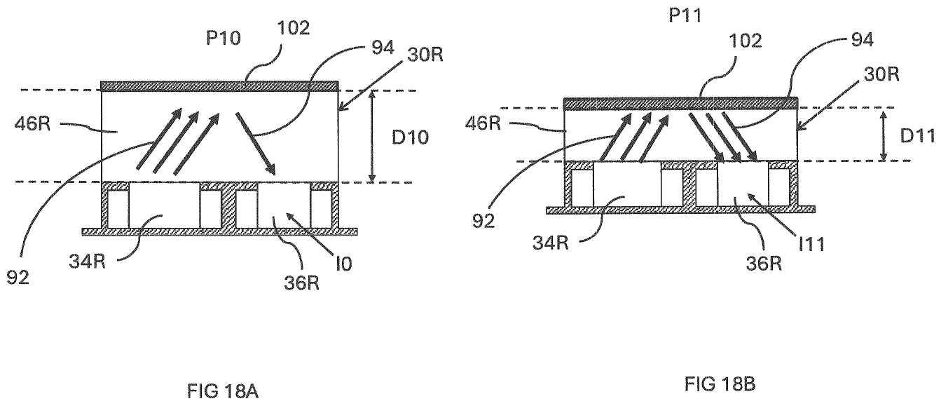

Referring to FIGS. 18A-18B, a sensor 30R can be substantially similar to the sensor 30V shown in FIGS. 17G-17K. The sensor 30R is also configured to detect pressure from an external source such as a finger 98 (FIG. 17L) substantially as previously described for sensor 30V (FIGS. 17G-17K). The sensor 30R includes a light source (LS) device 34R for emitting light 92 and a photodetector (PD) device 36R for detecting reflected light 94. The sensor 30R also includes a membrane light guide structure 46R having a variable thickness changeable as a function of pressure P10-P11 for varying the intensity of the reflected light 94 detected by the photodetector (PD) device 36R. The sensor 30R can also include a reflector 102 on the membrane light guide structure 46R. The reflector 102 can comprise a thin layer of a reflective or semi-reflective material, such as a semitransparent polymer, deposited or otherwise formed, on the membrane light guide structure 46R. The reflector 102 can comprise one or more light reflecting materials having a Young's modulus (E) selected such that the reflector 102 remains elastic when a known load is applied, substantially as previously described. It is important that under the maximum force, the reflector 102 still operates in the elastic regime and is not plastically deformed. The reflector 102 can comprise a single layer or a multilayer of reflective materials. For example, depending on the application, suitable materials for the reflector 102 include titanium/aluminum, molybdenum/gold, and tungsten/titanium stacking layers. The reflector 102 can also comprise a semitransparent or transparent polymer having reflective particles embedded therein.

As shown in FIG. 18A, an initial pressure can be P10 and the distance which corresponds to the thickness of the membrane light guide structure 46R can be D10. As shown in FIG. 18B, by applying a greater pressure P11, the distance can be decreased to D11. In addition, the intensity of the reflected light 94 can be correlated to the distance D11, with a lesser distance reflecting more light (e.g., D11<D10). Further, the reading of the light intensity I11 occurring with D11 is larger than that occurring with D10. By reading the amount of light intensity at the photodetector (PD) device 36R, a measurement of the pressure P10-P11 being applied can also be determined.

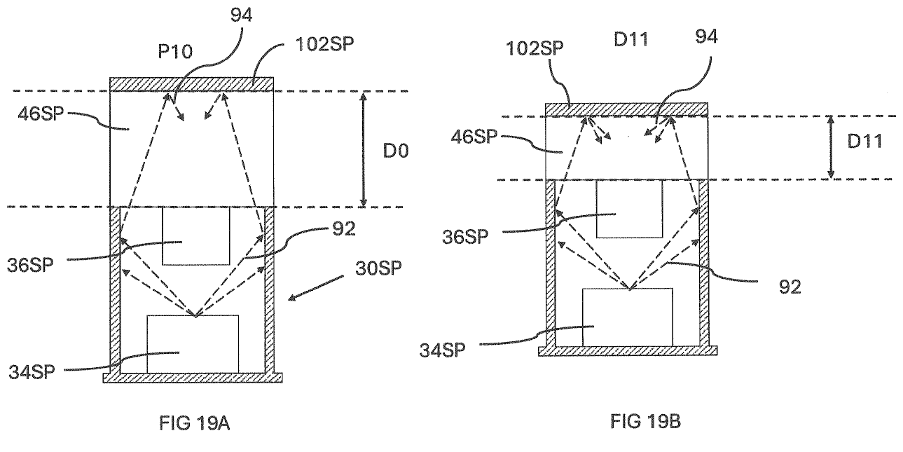

Referring to FIGS. 19A-19B, a sensor 30SP is substantially similar to the photoplethysmography (PPG) sensor 30 shown in FIG. 3A having a stacked configuration. The sensor 30SP is also configured to detect pressure substantially as previously described for sensor 30V (FIGS. 17A-17E). The sensor 30SP includes a light source (LS) device 34SP for emitting light 92 and a photodetector (PD) device 36SP for detecting reflected light 94 in a stacked configuration as previously described. The sensor 30SP also includes a membrane light guide structure 46SP having a variable thickness D0-D11 that can be changed as a function of pressure P0-P11 for varying the intensity of the reflected light 94 detected by the photodetector (PD) device 36SP. The sensor 30SP also includes a reflector 102SP on the membrane light guide structure 46SP, substantially as previously described for reflector 102 (FIG. 18A).

While a number of exemplary aspects and embodiments have been discussed above, those of skill in the art will recognize certain modifications, permutations, additions and subcombinations thereof. It is therefore intended that the following appended claims and claims hereafter introduced are interpreted to include all such modifications, permutations, additions and sub-combinations as are within their true spirit and scope.

What is claimed is:

1. A photoplethysmography (PPG) sensor comprising:
an integrated package comprising a light source (LS) device configured to emit light into biological tissue of a user and a photodetector (PD) device configured to receive reacted light transmitted through the biological tissue;
a plurality of metal interconnects electrically connected to connection pads on the light source (LS) device and the photodetector (PD) device;
an opaque covering structure configured to at least partially enclose the light source (LS) device and the photodetector (PD) device; and
a light guide structure inside the opaque covering structure comprising at least one non-opaque layer configured to guide the light emitted by the light source (LS) device;
the metal interconnects and the light guide structure configured to support the light source (LS) device and the photodetector (PD) device.

2. The photoplethysmography (PPG) sensor of claim 1 wherein the light guide structure includes a light escape area in light communication with the light source (LS) device, and a detection area in light communication with the photodetector (PD) device.

3. The photoplethysmography (PPG) sensor of claim 1 wherein the non-opaque layers of the light guide structure have a distributed Bragg reflector (DBR) configuration for light guiding.

4. The photoplethysmography (PPG) sensor of claim 1 further comprising a dam enclosing the detection area on the light guide structure configured to prevent stray light from entering the photodetector (PD) device.

5. The photoplethysmography (PPG) sensor of claim 1 wherein the light source (LS) device comprises a vertical light emitting diode (VLED) die having multiple light emitting zones.

6. A photoplethysmography (PPG) sensor comprising:
an integrated package comprising a light source (LS) device configured to emit light into biological tissue of a user and a photodetector (PD) device configured to receive reacted light transmitted through the biological tissue, the light source (LS) device and the photodetector (PD) device having a plurality of connection pads;
a plurality of metal interconnects electrically connected to the connection pads on the light source (LS) device and the photodetector (PD) device;
an opaque covering structure configured to enclose the light source (LS) device and the photodetector (PD) device; and
a light guide structure inside the opaque covering structure comprising at least one non-opaque layer configured to guide the light emitted by the light source (LS) device, the light guide structure having a light escape area in light communication with the light source (LS) device, and a detection area in light communication with the photodetector (PD) device.

7. The photoplethysmography (PPG) sensor of claim 6 wherein the opaque covering structure comprises an open end and at least four sides enclosing the light source (LS) device and the photodetector (PD) device.

8. The photoplethysmography (PPG) sensor of claim 6 wherein the light source (LS) device comprises a packaged (LED) die having a plurality of light emitting zones.

9. The photoplethysmography (PPG) sensor of claim 6 wherein the photodetector (PD) device and the light source (LS) device have a stacked configuration configured to minimize noise/crosstalk.

10. The photoplethysmography (PPG) sensor of claim 6 wherein the integrated package has the configuration of a chip scale package (CSP).

11. The photoplethysmography (PPG) sensor of claim 6 wherein the integrated package has the configuration of a package on package (POP) fan out chip scale package (FOCSP) having a (VCSEL) on driver (VoD) electrical configuration.

12. The photoplethysmography (PPG) sensor of claim 6 wherein the opaque covering structure includes a plurality of internal metal interconnects configured to rigidify the opaque covering structure.

13. The photoplethysmography (PPG) sensor of claim 6 further comprising a redistribution layer (RDL) on the light guide structure in electrical communication with the metal interconnects.

14. The photoplethysmography (PPG) sensor of claim 6 wherein the light source (LS) device is mounted to a solid closed end of the opaque covering structure, and the photodetector (PD) device is embedded in the light guide structure proximate to the open end of the opaque covering structure.

15. The photoplethysmography (PPG) sensor of claim 6 wherein the at least one non-opaque layer of the light guide structure includes a plurality of non-opaque layers having different refractive indexes and a distributed Bragg reflector (DBR) configuration.

16. The photoplethysmography (PPG) sensor of claim 6 further comprising a lens on the light guide structure.

17. The photoplethysmography (PPG) sensor of claim 6 further comprising a dam enclosing the detection area on the light guide structure configured to prevent stray light from entering the photodetector (PD) device.

18. The photoplethysmography (PPG) sensor of claim 6 wherein the non-opaque layers of the light guide structure have a distributed Bragg reflector (DBR) configuration for light guiding.

19. A photoplethysmography (PPG) sensor comprising:
an integrated package comprising a light source (LS) device configured to emit light into biological tissue of a user and a photodetector (PD) device configured to receive reacted light transmitted through the biological tissue, the light source (LS) device and the photodetector (PD) device having a plurality of connection pads;
a plurality of metal interconnects electrically connected to the connection pads on the light source (LS) device and the photodetector (PD) device;
an opaque covering structure configured to enclose the light source (LS) device and the photodetector (PD) device, the opaque covering structure having a closed end and an open end; and
a light guide structure inside the opaque covering structure comprised of one or more non-opaque layers having different refractive indexes configured to guide the light emitted by the light source (LS) device, the light guide structure including a light escape area in light communication with the light source (LS) device and a detection area in light communication with the photodetector (PD) device;
wherein the light source (LS) device is mounted to the closed end of the opaque covering structure, and the photodetector (PD) device is embedded in the light guide structure proximate to the open end of the opaque covering structure with the photodetector (PD) device stacked on the light source (LS) device.

20. The photoplethysmography (PPG) sensor of claim 19 further comprising a redistribution layer (RDL) on the light guide structure proximate to the open end of the opaque covering structure configured to provide circuit connections to internal interconnects in the opaque covering structure and to the connection pads on the photodetector (PD) device.

21. The photoplethysmography (PPG) sensor of claim 19 wherein optical axes of the light source (LS) device and the photodetector (PD) device are substantially aligned.

22. The photoplethysmography (PPG) sensor of claim 19 wherein the light source (LS) device comprises a plurality of packaged vertical light emitting diode (VLED) dice.

23. The photoplethysmography (PPG) sensor of claim 19 wherein the connection pads on the light source (LS) device and the photodetector (PD) device include anode connection pads and cathode connection pads and the metal interconnects include an external anode pad and an external cathode pad in electrical communication with the anode connection pads and the cathode connection pads.

24. The photoplethysmography (PPG) sensor of claim 19 further comprising a first dam enclosing the detection area on the light guide structure configured to prevent stray light from entering the photodetector (PD) device.

25. The photoplethysmography (PPG) sensor of claim 19 further comprising a second dam on the photodetector (PD) device enclosing a photodetector (PD) detection area on the photodetector (PD) device configured to prevent stray light from entering the photodetector (PD) device.

26. The photoplethysmography (PPG) sensor of claim 19 further comprising an addressed electrode on the integrated package in electrical communication with the metal interconnects configured to interconnect multiple photoplethysmography (PPG) sensors in an array.

27. The photoplethysmography (PPG) sensor of claim 19 wherein the light source (LS) device comprises a vertical light emitting diode (VLED) die having multiple light emitting zones.

28. The photoplethysmography (PPG) sensor of claim 19 wherein the light source (LS) device comprises a vertical light emitting diode (VLED) die having multiple light emitting zones paired with multiple photodetector (PD) devices.

29. The photoplethysmography (PPG) sensor of claim 19 wherein the light source (LS) device comprises a multiple color (LED) module configured to emit multiple colors of light.

30. The photoplethysmography (PPG) sensor of claim 19 wherein the light source (LS) device comprises a multiple color (LED) module configured to emit multiple colors of light paired with multiple photodetector (PD) devices.

31. A photoplethysmography (PPG) array comprising:
a plurality of photoplethysmography (PPG) sensors electrically connected in an array, each discrete photoplethysmography (PPG) sensor comprising an integrated package comprising:
a light source (LS) device configured to emit light into biological tissue of a user and a photodetector (PD) device configured to receive reacted light transmitted through the biological tissue;
a plurality of metal interconnects electrically connected to connection pads on the light source (LS) device and the photodetector (PD) device;
an opaque covering structure configured to at least partially enclose the light source (LS) device and the photodetector (PD) device; and
a light guide structure inside the opaque covering structure comprising at least one non-opaque layer configured to guide the light emitted by the light source (LS) device;
the metal interconnects and the light guide structure configured to support the light source (LS) device and the photodetector (PD) device;
the light source (LS) device and photodetector (PD) device including addressed electrodes in electrical communication in the array.

32. The photoplethysmography (PPG) array of claim 31 wherein the light guide structure includes a light escape area in light communication with the light source (LS) device, and a detection area in light communication with the photodetector (PD) device.

33. The photoplethysmography (PPG) array of claim 31 wherein the non-opaque layers of the light guide structure have a distributed Bragg reflector (DBR) configuration for light guiding.

34. The photoplethysmography (PPG) array of claim 31 further comprising a dam enclosing the detection area on the light guide structure configured to prevent stray light from entering the photodetector (PD) device.

35. The photoplethysmography (PPG) array of claim 31 wherein the light source (LS) device comprises a vertical light emitting diode (VLED) die having multiple light emitting zones.

36. A sensor comprising:
an integrated package comprising a light source (LS) device configured to emit light into biological tissue of a user and a photodetector (PD) device configured to receive reacted light transmitted through the biological tissue and reflected light from the light source (LS) device;
an opaque covering structure configured to at least partially enclose the light source (LS) device and the photodetector (PD) device; and
a light guide structure inside the opaque covering structure comprising at least one non-opaque layer configured to guide the light emitted by the light source (LS) device;
a membrane light guide structure on the light guide structure having a thickness that can be varied as a function of a pressure exerted on the membrane light guide structure such that a distance traveled by the reflected light can be varied.

37. The sensor of claim 36 wherein the membrane light guide structure comprises a compressible polymer configured to detect pressure.

38. The sensor of claim 36 wherein the light source (LS) device and the photodetector (PD) device have a side-by-side configuration.

39. The sensor of claim 38 further comprising a reflector on the membrane light guide structure configured to increase the reflected light, the reflector comprising a material having a Young's modulus selected such that the material remains elastic at the pressure.

40. The sensor of claim 39 wherein the material for the reflector comprises a reflective polymer, a metal stack or a polymer having reflective particles embedded therein.

41. The sensor of claim 36 wherein the light source (LS) device and the photodetector (PD) device have a stacked configuration.

42. The sensor of claim 41 further comprising a reflector on the membrane light guide structure configured to increase the reflected light, the reflector comprising a material having a Young's modulus selected such that the material remains elastic at the pressure.

43. The sensor of claim 42 wherein the material for the reflector comprises a reflective polymer, a metal stack or a polymer having reflective particles embedded therein.
